(19) United States
(12) Patent Application Publication
(10) Pub. No.: US 2008/0230081 A1
(43) Pub. Date: Sep. 25, 2008

(54) HORNY LAYER REMOVER

(76) Inventor: Jin-Ho MOON, Shiheung (KR)

Correspondence Address: EDWARDS ANGELL PALMER & DODGE LLP, P.O. BOX 55874, BOSTON, MA, 02205, US

(21) Appl. No.: 12/033,093

(22) Filed: Feb. 19, 2008

(30) Foreign Application Priority Data
Mar. 23, 2007 (KR) 10-2007-28396

(51) Int. Cl. A45D 29/00; A61B 17/54

(52) U.S. Cl. 132/75.6; 132/76.4; 606/131

(57) ABSTRACT

The invention is a horny layer remover with simple and sanitary structure to be easily made and to prevent the damage of skin and effectively remove the horny layers according to the hardness of the horny layer. The invention includes at least one blade in the shape of circle, curve or closed loop on one or both side of the body. And the blade is integrally formed with the body by means of injection molding.

### CROSS-REFERENCE TO RELATED APPLICATION

This application claims under 35 U.S.C. §119(a) the benefit of Korean Patent Application No. 10-2007-0028396 filed Mar. 23, 2007, the entire contents of which are incorporated herein by reference.

### BACKGROUND OF THE INVENTION

The present invention relates to a horny layer remover, and more particularly to a horny layer remover with simple and sanitary structure to be easily manufactured and to prevent the damage of skin and remove the horny layers effectively according to the hardness of the horny layer.

Generally, when man wears shoes made of hard material and walks for a long time, the heel skin of his foot may be hardened to be a horny layer or a stratum corneum. This horny layer of the body can be formed on foot, hand or other portion of body owing to many other reasons. The horny layer may grow to be thick and cracks may be formed on the surface of the horny layer, which may incur pains and virus infections.

To remove the horny layer of skin, various horny layer removers have been used. The conventional horny layer removers comprise a body, a grinding plate attached on the body and a handle extending from the body. The grinding plate is bonded with grinding particles such as sand, stone or metal particle. To use this horny layer remover, the user may grip the handle of the horny layer remover and grinds the horny layer by the grinding plate.

But, the horny layer removers are rather complicate in structure because it comprises two parts of the body and the grinding plate, therefore the manufacturing process of it may be complicate and the manufacturing cost may be high. Moreover, as the user grinds the horny skin by means of the grinding plate, the normal skin adjacent to the horny layer may be hurt. And during the grinding, fine pieces of horny layer or impurities may remain in the grinding plate, which may not be easily removed or washed and it may bring about sanitary problems. And according to the form and the hardness of the horny layer, different horny layer remover with proper grinding plate should be used, which may be troublesome.

The invention is suggested to solve the above problems, and the object of the invention is to provide a horny layer remover with simple and sanitary structure to be easily made and easily washable, and to remove the horny layers effectively according to the hardness of the horny layer without skin damage.

### SUMMARY OF THE INVENTION

According to an aspect of the invention, there is provided a horny layer remover which includes at least one blade in the shape of circle, curve or closed loop on one or both side of the body.

According to other aspect of the invention, the blade is integrally formed with the body by means of injection molding.

According to other aspect of the invention, the blade is formed with the material different to that of the body.

According to other aspect of the invention, the blade is formed on one side of the body and a recess is formed on the other side of the body.

According to other aspect of the invention, a part of the blade is formed in the shape of the tooth.

According to other aspect of the invention, the blades are formed in the shape of the multilayered concentric circle or annual ring.

According to other aspect of the invention, the blade is formed on one side of the body a brush or a grind stone is provided on the other side of the body.

According to the invention as described above, as the blades 2 of the shape of circle, curve or closed loop are formed on one or both side of the body 1, the straight direction of the reciprocating movement of the horny layer remover is not equal to the line of the curved blades, so the skin adjacent to the horny layer area may not be cut or damaged by the blade. And as the blades 2 and the body 1 are formed integrally by means of injection molding, the structure and the manufacturing process can be simplified. And if the blade 2a is made of metal having the higher hardness than the blade 2 and it is attached to the body 1, the blades 2, 2a may selectively used according to the hardness of the horny layer to achieve effective removing of the horny layer. And at least a part of the blades 2, 2a are formed to have teeth, so the horny layer may be easily removed. And as the gap between the blades 2, 2a have considerable space, the particles of removed skin or impurities may be easily washed or cleaned, which is more sanitary than the conventional remover in which the particle of the removed skin or impurities are attached on the grinding particles such as sand or stone firmly.

In addition, as the recess 3 is formed on the other side of the body 1, it can be used as the tray or container for a soap cake or the like in bathroom. And as the brush 6 is attached on the recess 3, the brush 6 may used to remove the dust from the skin or clothes. And a grind stone 4 may be attached on the recess 3, which may be selectively used to remove the horny skin according to the hardness of the horny skin.

### BRIEF DESCRIPTION OF THE DRAWINGS

The above and other objects or advantages of the present invention will become more apparent by describing the preferred embodiments thereof in detail with reference to the accompanying drawings in which;

FIG. 1 is an exploded view of first embodiment of the horny layer remover according to the invention,

FIG. 2 is a side section of the above first embodiment of the invention,

FIG. 3 is an exploded view of the second embodiment of the invention,

FIG. 4 is an exploded view of the third embodiment of the invention,

FIG. 5 is a side view of the fourth embodiment of the invention.

### DETAILED DESCRIPTION OF THE PREFERRED EMBODIMENT

A preferred embodiment of the invention will be described with reference to the accompanying drawings. Like reference numerals denote like elements through the following embodiments.

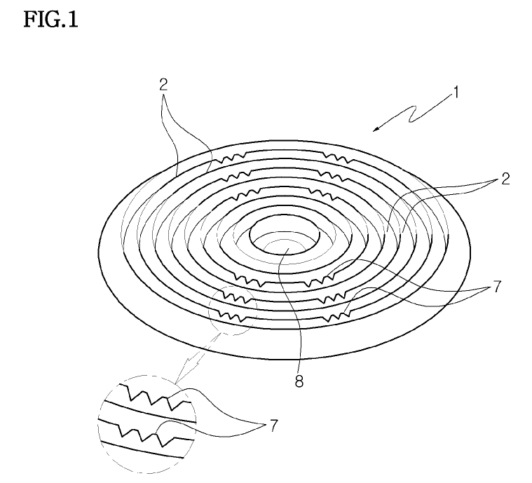

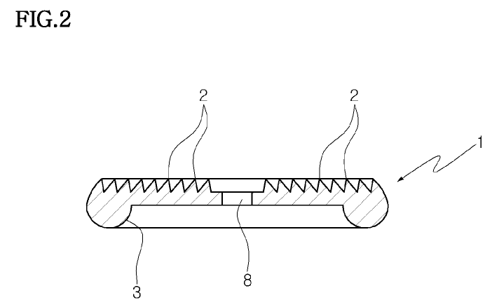

FIG. 1 and FIG. 2 show the first embodiment of the horny layer remover according to the invention. As shown, the horny layer remover comprises a circular body 1, and a plurality of the concentric blades 2 are integrally formed on the surface of the body 1 by means of injection mold. The structure of this type is simpler than that of the conventional horny layer remover in which a separate grinding plate is attached on the body.

The section of the blade according to the embodiment is shown such that the inclined surface of the blade 2 facing the center of the body 1 is steeper than that of the other side of the blade 2. The inclination of the surfaces of the blades 2 may be differently determined to make the blade 2 sharper or smoother, which may be properly selected to the conditions such as the form or hardness of the horny layer. And as the line of the blade 2 is curved, it may not cut or damage the skin during the straight movement of the horny layer removing operation.

Preferably, at least a part of the blades 2 may have the teeth, which may be advantageous to remove the relatively harder horny layer easily. And as the recess 3 is formed on the other side of the body 1, it can be used a tray or container for soap cake or the like if it is disposed upside down. A hole 8 is formed on the center of the recess 3, which may function as a drain hole for the soap cake tray.

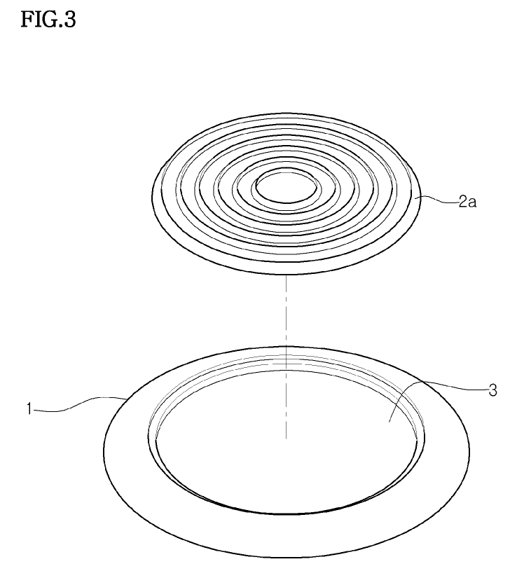

FIG. 3 shows another embodiment of the invention, in which the concentric circular blades 2a are made by a separate body of the different materials such as aluminum, stainless steel or other metal. The body of the blades 2a is attached on the recess 3 of the body 1. If the horny layer remover includes metal blades 2a with high hardness on one side together with the plastic blades 2 with low hardness on the other side, then the proper blades 2 or 2a may be selected according to the hardness of the horny layer.

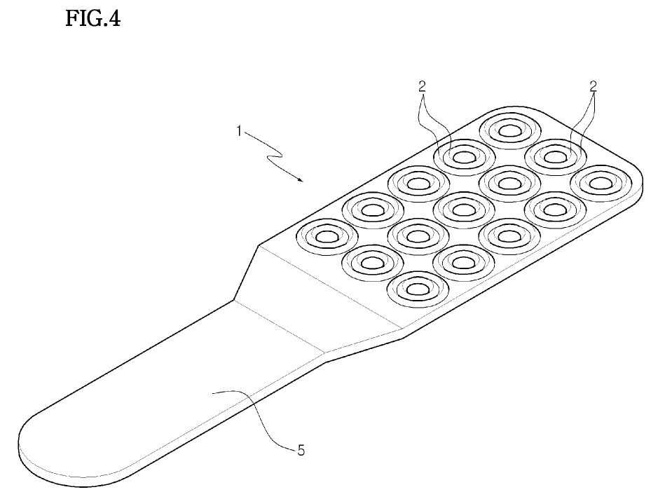

FIG. 4 shows another embodiment of the invention, in which a plurality of the concentric layers 2, 2a are disposed on the body 1, and a handle 5 is formed on one side of the body 1. This embodiment of the invention may be easily made by means of injection mold, and the user may use it conveniently by gripping the handle 5.

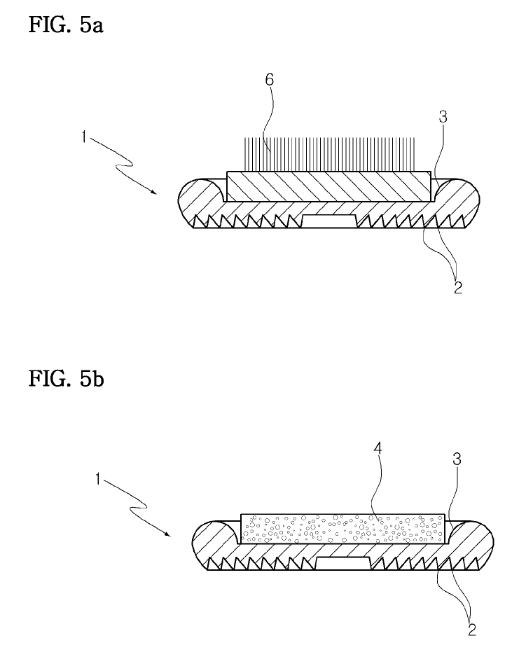

FIG. 5a and FIG. 5b show other embodiments of the invention. As shown in FIG. 5a, blades 2, 2a are formed on one side of the body 1, and a brush 6 is attached on the recess 3 of the other side of the body 1. The brush 6 may clean the skin particles or the impurities from the horny layer after the horny layer is grinded. And, as shown in FIG. 5b, a conventional grind stone 4 may be attached on the recess 3 of the body 1, which may be softer than the blades 2, 2a, and could be selectively used according to the hardness of the horny layer.

Though the preferred embodiments are described to have concentric blades 2, 2a, the shape of blades 2, 2a may be properly selected from the shapes which includes as ellipse, oval, arch, other type of closed looped curve or polygonal. If the blades 2, 2a have the shape of segmented curve, the ends of the segmented curves are preferably connected to the projected edge part of the body, so that the ends of the segmented curve may not cut or damage the skin of the user.

1. A horny layer remover which includes at least one blade in the shape of circle, curve or closed loop on one or both side of the body.

2. A horny layer remover according to claim 1, wherein the blade is integrally formed with the body by means of injection molding.

3. A horny layer remover according to claim 1, wherein the blade is formed with the material different to that of the body.

4. A horny layer remover according to claim 1 to 3, wherein the blade is formed on one side of the body and a recess is formed on the other side of the body.

5. A horny layer remover according to claim 1 to 3, wherein, at least a part of the blade is formed in the shape of the tooth.

6. A horny layer remover according to claim 1 to 3, wherein the blades are formed in the shape of multilayered concentric circle or annual ring.

7. A horny layer remover according to claim 1 to 3, wherein blade is formed on one side of the body a brush or a grind stone is provided on the other side of the body.
